(19) United States
(12) Patent Application Publication
(10) Pub. No.: US 2023/0381487 A1
(43) Pub. Date: Nov. 30, 2023

(54) VENTRICULAR ASSIST SYSTEM AND METHOD

(71) Applicant: Cardiobionic Pty Ltd, Cronulla, NSW (AU)

(72) Inventors: Peter Joseph Ayre, Frenchs Forest (AU); Reginald Brian Haddrell, Cronulla (AU); Ross James Dean, Cronulla (AU)

(73) Assignee: Cardiobionic Pty Ltd, Cronulla, NSW (AU)

(21) Appl. No.: 18/032,032

(22) PCT Filed: Oct. 15, 2021
(86) PCT No.: PCT/AU2021/051210

§ 371 Date: Apr. 14, 2023

(30) Foreign Application Priority Data
Oct. 15, 2020 (AU) 2020903749

(51) Int. Cl. A61M 60/117; A61M 60/183; A61M 60/546; A61M 60/876; A61M 60/878; A61M 60/88

(52) U.S. Cl. CPC A61M 60/117; A61M 60/183; A61M 60/546; A61M 60/876; A61M 60/878; A61M 60/88; A61M 2205/3334; A61M 2230/04

(57) ABSTRACT

The present invention relates to provides a heart assist device comprising one or more blood pumps for connection to a heart, a control module for controlling operation of the one or more pumps, a power module for powering the operation of the control module and the one or more pumps, and a lead for supplying power between the control module and the one or more pumps. The heart assist device includes a control module adapted for controlling operation of one blood pump, in a left ventricular assistance device configuration in or a right ventricular assistance device configuration, or for controlling two blood pumps in a bi-ventricular assistance device configuration.

### TECHNICAL FIELD

The present invention relates to heart assist devices and systems commonly referred to as ventricular assist devices and to associated sensors, controls systems and power supplies.

### BACKGROUND OF INVENTION

Heart assist devices are mechanical circulatory support devices that are used in the management of advanced heart failure. Such devices are increasingly being used as the number of heart failure patients continues to rise and the number of available donor organs does not meet this need.

Left ventricular assist devices (LVAD) are a relatively common form of heart assist device is and they are used if the patient suffers from severe unrecoverable left ventricular function. These devices are typically used as a bridge to transplant therapy. However, these devices are increasingly being considered as a destination therapy meaning the patient would live with the device for the rest of their life. This can be due to shortages of donor organs or because the patient is not a candidate for heart transplant.

Typical LVAD devices in use include an inflow cannula inserted into apex of the left ventricle through which blood enters the pump and is delivered to the ascending aorta by means of an outflow cannula. The LVAD works in parallel with the native heart with the aortic valve opening due to intrinsic contraction of the heart. A driveline is connected to an external controller with battery packs the patient must have with them at all times. Improvements in device technology means smaller devices are being developed that don't occupy the abdominal cavity which decreases infection risk.

The LVAD settings are set by the device operator to optimise left ventricular unloading to improve cardiac output. Parameters of the pump include the speed of the pump, the flow rate of the pump and the power consumption of the pump.

Speed, measured in RPM of the impeller of a centrifugal pump, is often the only parameter of the pump the operator has control over. A speed is chosen at which the patient is not symptomatic with respect to their heart failure while also allowing the aortic valve to open. This means that the native heart is also contributing to cardiac output in addition to what the pump is supporting.

Flow measured in Litres/minute reflects cardiac output and can also be affected by systemic blood pressure and blood viscosity through the LVAD itself.

Power measured in Watts is the direct measurement of energy required by the device to pump blood at the set RPM. Any deviation from these settings can trigger an alarm on a controller and may be communicated to the patient's device management team of physicians.

Pharmacologic therapies are used in conjunction with LVAD device therapy. These include diuretic agents, beta blockers, Angiotensin-converting-enzyme (ACE) inhibitors, Angiotensin Receptor Blockers (ARB), angiotensin receptor-neprilysin inhibitor (ARNI) and aldosterone antagonists. Additionally the increased risk of thrombal embolic events means Warfarin and aspirin are typically used to mitigate the risk of complications due to the patient becoming hypercoagulable due to increased inflammation and contact and tissue factor initiated coagulation and platelet activation.

Bleeding is the most common complication after LVAD implantations with common sites being the surgical site and gastrointestinal bleeding. Concerning infections are those that are related to the LVAD components themselves including infections related to the implanted pump components or the percutaneous driveline. Pump thrombosis in the pump itself or in the outflow cannula which can lead to circulatory failure. Stroke is also a complication that can be related to use of warfarin.

The heart assist device can either implanted or can be external to the body. Commonly the blood pump device itself includes a rotary pump such as an axial flow pump, a centrifugal pump or a mixed flow pump that can either be powered electrically or pneumatically. Such rotary pumps can have valves to prevent back flow.

However, other types of pumps exist such as sac-type pumps where blood is squeezed from a diaphragm electromagnetically or pneumatically. These sac-type pumps usually contain valves to prime the pump, produce forward flow and to prevent back flow. However, the rotary pumps are usually more efficient, smaller and lighter than diaphragm pumps.

The most common type of heart assist device used presently is the centrifugal blood pump which is implanted and that support the left side of the heart only. These are called left Ventricular assist devices or LVADs. Approximately 40% of recipients of implanted LVADs will develop right side heart complications after the LVAD is implanted.

Another type of device used in the management of advanced heart failure which to date has had limited success is the heart replacement device, also known as total artificial heart (TAH) therapy. This is where the native heart is completely removed and a mechanical device is substituted. This therapy is considered high risk as the safety net of the native heart is removed and normal neural and chemical feedback mechanisms connecting the heart and the brain become void.

Yet another type of device used in the management of advanced heart failure is a Bi-Ventricular Assist Device or BIVAD. A BIVAD supports both sides of the heart and is usually made up of an LVAD and/or an RVAD (right ventricular assist device). For BIVAD systems, surgeons presently use two LVAD systems where the patient has to carry a large amount of controller accessories. The exit site wounds are large because of large cannula and/or multiple drivelines which increases infection risk. Some BIVAD arrangements are large temporary pneumatic systems with bulky drive consoles, which means the patient cannot leave hospital easily.

The discussion of the background to the invention included herein including reference to documents, acts, materials, devices, articles and the like is included to explain the context of the present invention. This is not to be taken as an admission or a suggestion that any of the material referred to was published, known or part of the common general knowledge in Australia or in any other country as at the priority date of any of the claims.

### SUMMARY OF INVENTION

Accordingly, in one aspect, the present invention provides a heart assist device comprising: one or more blood pumps for connection to a heart; a control module for controlling operation of the one or more pumps; a power module for powering the operation of the control module and the one or more pumps; and a lead for supplying power between the control module and the one or more pumps.

In embodiments, described in further detail below, the heart assist device includes a control module adapted for controlling operation of one blood pump, in a left ventricular assistance device configuration in or a right ventricular assistance device configuration, or for controlling two blood pumps in a bi-ventricular assistance device configuration. The control module preferably includes an external controller device that is located outside of the patient's body and in embodiments includes an implanted controller. In embodiments, the processing and control operations of the control module are distributed between the external and the implanted controller. In embodiments, the external controller exhibits complete control over the operations of the left and/or right pumps including for estimating the flow rate of the pumps individually, determining the power drawn by the pumps and the speed of the pumps from sensors, such as hall effect sensors, or back EMF speed detection of the speed of the pumps. In embodiments, the external controller includes two processors for controlling the operation of the two pumps, for example for dynamically controlling the power supplied to the pumps, and a third processor for controlling the operation of the controller. The third processor is configured for receiving user inputs from a remote or central computing platform operated by a medical practitioner, such as a cardiologist, to program the configuration of the controller according to the mode of application and the desired ventricular assistance therapy being employed (e.g. LVAD, RVAD, BIVAD), and for conducting other operations including estimating pump flow rates and flow balancing between the pumps.

Advantages of the aforementioned embodiments, which are described in further detail herein below, include providing a modular and flexible heart assist device that can be configured by a surgeon and/or a cardiologist for LVAD therapy only, RVAD therapy only or for BIVAD therapy. When a surgeon is conducting very invasive heart surgery on a patient to implant pumps and conduits into the heart and major blood vessels such as the aorta and the pulmonary artery, the needs of the patient for an LVAD, RVAD or BIVAD therapy can dynamically change and embodiments of the invention provide a modular and configurable platform to provide each kind of ventricular assistance therapy as required. Furthermore, the control module facilitates programmable control over the left and right pumps in a BIVAD configuration and flow balancing therebetween.

In embodiments, at least part of the control module is located externally and the one or more pumps are implantable in the body and the lead extends percutaneously between the external part of the control module and the one or more implanted pumps.

Preferably, the control module includes one or more processors for controlling the operation of the one or more pumps.

Preferably, the processors for controlling the operation of the one or more pumps are distributed between an external part of the control module and an implanted part of the control module.

In embodiments, the control module independently and simultaneously controls the speed of each of the pumps to thereby control the output of each pump.

In embodiments, the control module is operable to estimate the flow through each of the one or more pumps from data received at the control module, which data is indicative of electrical power provided to each of the one or more pumps and/or a speed of each of the one or more pumps.

In embodiments, the control module controls the speed of each of the one or more of the pumps according to the estimate of flow through each of the one or more pumps.

In embodiments, the control module controls the speed of each of the one or more pumps to increase the output of each of the one or more pumps during the systole phase of the cardiac cycle and to reduce the output during the diastole phase of the cardiac cycle.

In embodiments, the heart assist device is configured to include one or more hall effect sensors in the one or more of the pumps and the control module is configured to receive signals from the hall effect sensors and to determine the speed of the one or more of the pumps. In other embodiments, the heart assist device is configured to determine the speed of the pumps with back EMF signal detection and processing.

In embodiments, the control module can be configured to receive signals from an ECG sensor and/or an accelerometer sensor implanted on or near the heart, indicative of the natural electrical activity and/or movement of the heart.

In embodiments, the control module controls the speed of each one of the pumps to be synchronised with the systole and diastole phases of the natural heart indicated in the ECG sensor signals.

In embodiments, the control module executes an algorithm to determine the systole and diastole phases of the natural heart represented in the ECG sensor signals.

In embodiments, the control module is configured to control the speed of each of the one or more pumps to balance the flow through the left and right ventricles.

In embodiments, the control module is configured to set a target speed for a one of the pumps associated with the right ventricle to generate a flow equivalent to a proportion of the flow of a one of the pumps associated with the left ventricle.

Preferably, the proportion is between about 85 and 95 percent or any increment therebetween or is preferably 90 percent.

In embodiments, the power module includes at least one battery that is connected to the control module.

In embodiments, the power module includes at least one external battery and an implanted battery.

In embodiments, the lead includes an implanted portion connected to the one or more pumps and an external portion connected to an external part of the control module and an electrical connection between the implanted and external portions of the lead.

In embodiments, the electrical connection includes an implanted connector that is rigidly connectable to the iliac crest of the pelvis and that is adapted for electrical connection with an external connector that is connected to the external portion of the lead.

In embodiments, the implanted connector has a percutaneous portion that passes through the skin and an external electrical contact for connection with an electrical contact of the external connector.

In embodiments, the control module is configured to send and receive data to a remote processing device via either a wired or a wireless connection such as Bluetooth, Wi-Fi or a mobile telecommunications network.

In embodiments, the remote processing device is adapted to communicate with the control module to adjust parameters for the control of the one or more pumps by the control module.

In embodiments, a connector assembly is provided for connecting a flow inlet of one of the blood pumps to a chamber of a heart.

In embodiments, the connector assembly includes a first fitting for implantation within an opening through a wall of a heart chamber, the first fitting including an axial passage for receiving a second fitting connected to the flow inlet of the one or more blood pumps.

Preferably, the second fitting is connected indirectly to the flow inlet of the one of the blood pumps with an elongated conduit for locating the pump in a suitable location distally from the heart.

Preferably, the second fitting is connected directly to a housing of the one of the blood pumps at the flow inlet for locating the pump immediately adjacent to the heart.

In embodiments, the first fitting is mounted within a cuff adapted to be sutured into place circumferentially within the opening in the heart wall.

In embodiments, the cuff is formed of a surgical felt material.

In another aspect, the invention provides a heart assist device including: two blood pumps for connection to a heart for right and left ventricular assistance, wherein the two pumps are implanted in the body of a patient, a control module and a power module located externally of the body of the patient, the control module for controlling operation of the pumps and the power module for powering the operation of the pumps; and a lead extending from the pumps for percutaneous electrical connection between the pumps and the control module and the power module

In another aspect, the invention provides a heart assist device including,
a housing defining a chamber and including an inlet and an outlet;
an impeller contained within the chamber and configured to rotate about an axis; wherein the impeller includes a casing defining one or more cavities;
one or more permanent magnets contained within the one or more cavities;
wherein the housing includes first and second sets of electric coils on axially opposite sides of the impeller,
wherein the electric coils are adapted to generate an electric field to drive the rotation of the impeller.

In embodiments, the casing is formed out of two parts that are sealed together to enclose the permanent magnets therewithin.

In embodiments, wherein a pair of first and second permanent magnets are stacked one upon the other within each blade of the impeller, wherein the dipoles of the first and second permanent magnets are oriented angularly to the axis of rotation of the impeller.

In embodiments, the one set of electric coils are in a base of the chamber and another set of electric coils are in a cover of the chamber.

In embodiments, the set of electric coils are mounted in the housing and are oriented angularly to the axis of rotation of the impeller.

In another aspect, the invention provides a control system for a bi-ventricular assistance device that includes two blood pumps for providing left and right ventricular assistance therapy, the system including:
a control module including a processor receiving data indicative of electrical power provided to the pump and/or a speed of the pump; and
estimating the flow through each one of the pumps from the data received at the processor by comparing the received data with a mathematical model indicative of the relationship between the electrical power supplied to the pump and/or the speed of the pump and the flow through the pump.

In embodiments, wherein the mathematical model is derived from measurements of flow through the pump with varying electrical power supplied to the pump and/or speeds of the pump.

In embodiments, the control module controls the output of the pumps by adjusting the electrical power provided to the pumps.

In embodiments, the processor is configured to control the output of the pumps to balance the flow through the left and right ventricles.

In embodiments, the processor is configured to set a target speed for the one of the pumps associated with the right ventricle to generate a flow equivalent to a proportion of the flow of the other one of the pumps associated with the left ventricle, wherein the proportion is between 85 and 95 percent or any increment therebetween or preferably 90 percent.

In another aspect, the invention provides a method for controlling a bi-ventricular assistance device that includes two blood pumps for providing left and right ventricular assistance therapy, the system including:
receiving at a processor of a control module data indicative of electrical power provided to the pump and/or a speed of the pump;
estimating the flow through each one of the pumps by comparing the received data with a mathematical model indicative of the relationship between the electrical power supplied to the pump and/or the speed of the pump and the flow through the pump.

In embodiments, the speed of the speed of the pumps is determined with back EMF signal detection and processing or with one or more sensors including a hall effect sensor embedded in the housing of the one or more pumps.

In embodiments, the method includes controlling the output of the pumps by adjusting the electrical power provided to the pumps.

In embodiments, the method includes controlling the output of the pumps to balance the flow through the left and right ventricles.

In embodiments, the method includes setting a target speed for the one of the pumps associated with the right ventricle to generate a flow equivalent to a proportion of the flow of the other one of the pumps associated with the left ventricle, wherein the proportion is between 85 and 95 percent or any increment therebetween or 90 percent.

These and other aspects and embodiments of the invention will become apparent from the foregoing summary of the drawings and the detailed description.

### BRIEF DESCRIPTION OF DRAWINGS

The present invention will now be described in more detail with reference to embodiments of the invention illustrated in the figures, wherein:

FIGS. 1 to 5 are diagrammatic illustrations of a variety of embodiments of an implantable left and/or right ventricular assist system in accordance with embodiments of the invention;

FIG. 5 is a diagrammatic illustration of an embodiment of an implantable left and/or right ventricular assist system including a percutaneous iliac crest connection assembly;

FIGS. 6a to 6d illustrate diagrammatically embodiments of a connector assembly for connecting a tube extending from a flow inlet of one of the blood pumps to a chamber of the heart;

FIGS. 7 to 9 are diagrammatic illustrations of embodiments of a left and/or right ventricular assist system in which the pumps are located externally;

FIG. 10 illustrates graphs plotting pressure versus flow for implanted left and right ventricular assist device pumps;

FIG. 11 illustrates graphs plotting pressure, flow rate, pump speed and current over time for a left ventricular assist device;

FIG. 12 illustrates schematically representations of the natural electrical impulses of the heart, the respective contraction and relaxation of the chambers of the heart and how signals from sensors such as an ECG sensor and/or an accelerometer sensor implanted on or near the heart assists in facilitating control of the operation of the pumps in accordance with embodiments of the invention;

FIG. 13 illustrates an isometric view of an embodiment of the left pump and the right pump;

FIG. 14 illustrates a top view of the pump of FIG. 13;

FIGS. 15 to 19 illustrate an embodiment of an impeller for the left pump or the right pump; and

FIG. 20 illustrate a side view of a frontal cross-section of the pump of FIG. 13 illustrating the housing, the chamber and the impeller contained therein.

### DETAILED DESCRIPTION

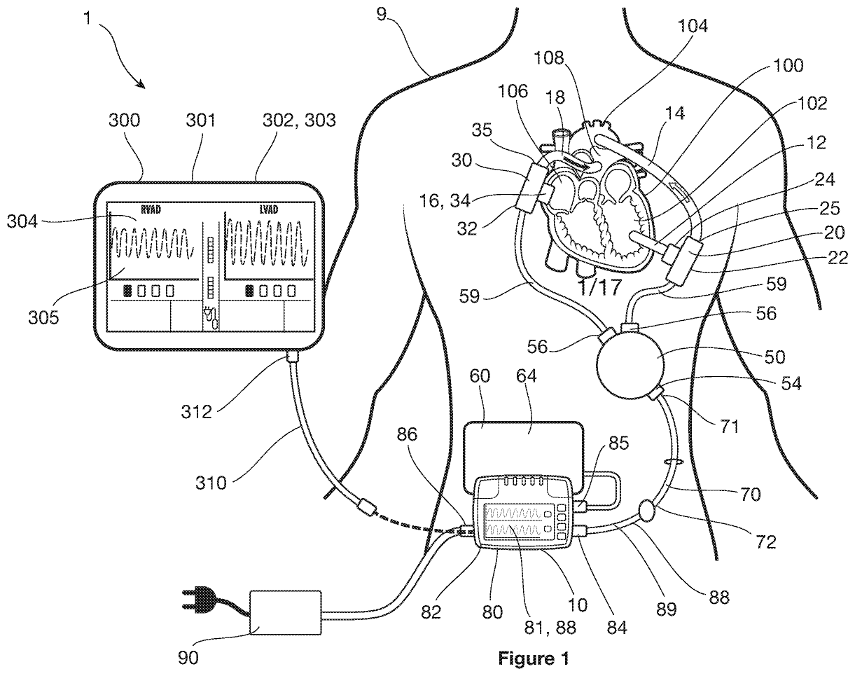

In one aspect, and with reference to FIGS. 1 to 6, the invention relates to a heart assist system 1 adapted for providing mechanical circulatory support for use in the management of advanced heart failure. In embodiments, the system 1 includes a heart assist device 10 comprising: one or more blood pumps 20, 30 for connection to a heart 100. A control module, which includes an external controller 80 and an implantable controller 50 for controlling operation of the one or more pumps 20, 30. A power module 60, which includes a rechargeable battery for powering the operation of the external controller 80 and the internal controller 50 and the one or more pumps 20, 30. A percutaneous lead 70 connected between the external controller 80 and the one or more pumps, via the implanted controller 50, and includes a conductor 72 for power and/or data transfer. Some components of the heart assist device 10 are adapted for implantation into a patient 9. As will be apparent, the control module can include a configuration of distributed processing and control between the external controller 80 and the internal controller 50.

In some embodiments, the implanted controller 50 can include no on-board processor and all of the processing and control of the pumps 20, 30 is exhibited by the external controller 80. In other embodiments, the implanted controller 50 includes one or more processors for control of the pumps 20, 30 and the external controller 80 includes one or more processors for controlling the operations of the external controller 80, including operation of a display and a user interface and for interfacing with a remote monitoring platform such as a separate computing device operated by a user such as a medical professional.

FIGS. 1 to 6 illustrate a variety of embodiments of the heart assist system 1. In particular, FIGS. 1 to 5 illustrate embodiments comprising a modular arrangement in which the two pumps 20, 30 are contained in separate housings 22, 32. In FIGS. 1, 2 and 5 the two housings 22, 32 are located separately from each other wherein a first one of the pumps 20 is located proximally to the left ventricle 102 of the heart 100 and configured for left ventricular assistance threrapy and the other one of the pumps 30 is located proximally to the right atrium 106 or ventricle of the heart 100 for right ventricular assistance therapy. Although in the Figures the right ventricular assistance pump 30 is illustrated as being implanted through the wall of the right atrium 106 it is to be appreciated that implantation may be through the wall of the right ventricle of the heart 100 as determined by the surgeon and cardiologist.

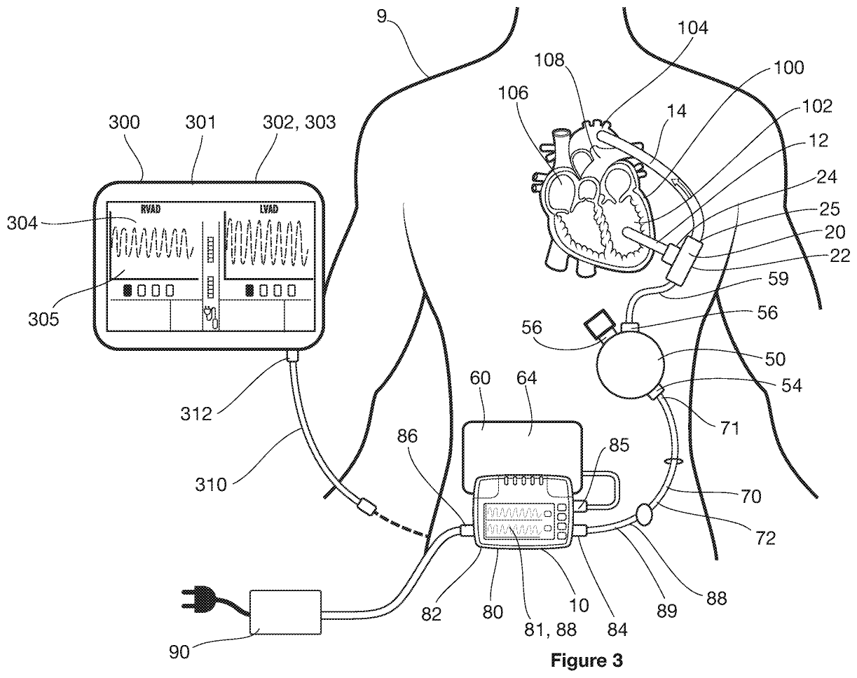

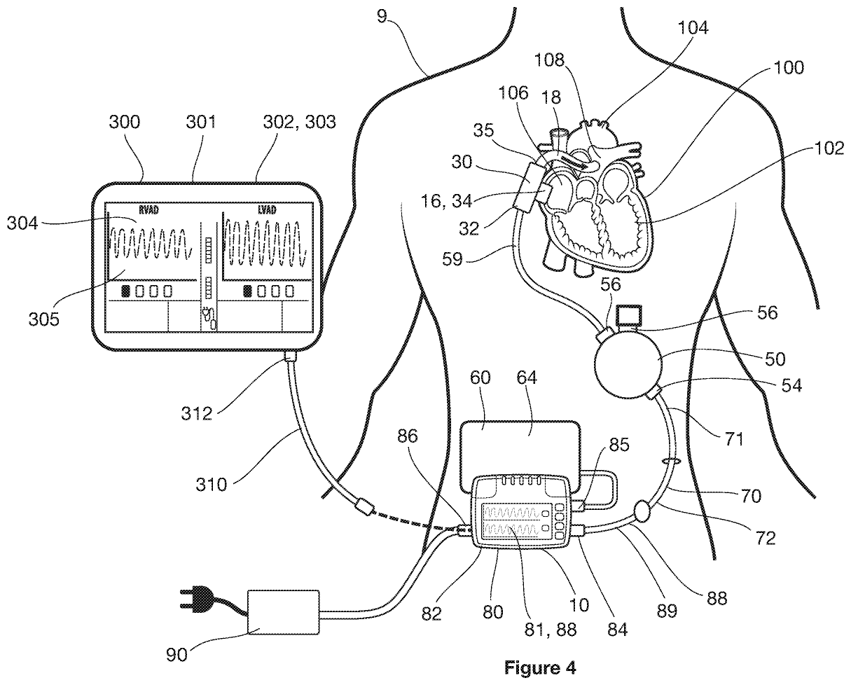

FIG. 3 illustrates an embodiment of the heart assist system 1 including only one of the pumps 20 which is located proximally to the left ventricle 102 of the heart 100 and is configured for left ventricular assistance therapy only. One of the inputs and outputs 54 of the implanted controller 50 is connected to the external controller 80 and one of the inputs/outputs 56 is connected to the implanted pump 20 and the other available input/output of 56 of the implanted controller 50 is not connected and is sealed with a cover. FIG. 4 illustrates another embodiment of the heart assist system 1 also including only a single one of the pumps 30 located proximally to the right atrium 106 and ventricle of the heart 100 for right ventricular assistance therapy only. One of the inputs and outputs 54 of the implanted controller is connected to the external controller 80 and one of the inputs/outputs 56 is connected to the implanted pump 30 and the other available input/output of 56 of the implanted controller 50 is not connected and is sealed with a cover. Accordingly, embodiments of the implanted controller 50 are configurable for implantation and for LVAD or RVAD or BIVAD therapy as required.

FIGS. 1, 2, 3 and 5 illustrate embodiments of the system 1 including an arrangement of the first pump 20 configured for left ventricular assistance therapy in which a tube 12 carries blood from the left ventricle 102 of the heart 100 to an inlet 24 of the housing 22 of the left ventricle pump 20. The pump 20 delivers blood through another tube 14 connected to an outlet 25 of the housing 22 to the aorta 104 that leads out to the body from the heart 100 and which delivers blood to the body.

FIGS. 1, 2, 4 and 5 illustrate embodiments of the system 1 including an arrangement of the second pump 30 configured for right ventricular assistance therapy in which a tube 16 carries blood from the right ventricle 106 of the heart 100 to an inlet 34 of the housing 32 of the right ventricle pump 30. The pump 30 delivers blood through another tube 18 connected to an outlet 35 of the housing 32 to the pulmonary artery 108 that leads from the heart 100 to the lungs.

Each of the embodiments of the heart assist system 1 of FIGS. 1 to 6 share in common a number of features. In particular, they each include the control module including the implantable module 50 and the external control module 80 for controlling operation of the one or more pumps 20, 30. Embodiments of the controller module will be described in further detail below. Furthermore, each of the embodiments of the heart assist system 1 of FIGS. 1 to 5 include the percutaneous lead 70 which is connected to the implanted module 50 and includes the conductor 72 for power and data transfer. Embodiments of the percutaneous lead 70 will be described in further detail below.

The external controller 80 includes a casing 82 housing internal components of the external controller 80 including one or more processors 83, a data storage device such as a solid state drive, a circuit board 87 and data and electrical inputs and outputs 84, 85, 86. The external controller 80 also includes a display 81 and a user interface 88 such as a keyboard or touchscreen. The external controller 80 also includes a receiver 89 to receive signals from a remote or central computing platform operated by a medical practitioner, such as a cardiologist, to program the configuration of the controller 80 according to the mode of application and the desired ventricular assistance therapy being employed (e.g. LVAD, RVAD, BIVAD).

The external controller 80 includes a pair of the processors 83 for controlling a respective one of the pumps 20, 30, namely by dynamically controlling the supply of power to the pumps 20, 30 from the external power supply 60 according to the selected therapeutic parameters. The external controller 80 also includes a third processor 83 for controlling other operations of the controller 80, namely the display 81, the user-interface 88, communication with the remote or central computing platform operated by a medical practitioner, such as a cardiologist. The third processor additionally or alternatively communicates with the other two pump control processors 83 to respectively transmit and receive data indicative of the operation of the pumps 20, 30 (e.g. pump speed and/or power consumption), to estimate the flow through each one of the pumps 20, 30 according to any of the methods described herein, and in real time to dynamically control the output of the pumps 20, 30 according to any of the methods described herein. Accordingly, the third processor 83 sits between and controls the operation of the two processors 83 that control operation of each of the individual pumps 20, 30.

In embodiments the implantable controller 50 includes a casing 52 formed out of a biocompatible material. The casing 52 houses internal components including one or more processors 53, a storage device 55 such as a solid state drive, a circuit board 57 and data and electrical inputs and outputs 54, 56. In other embodiments, the implantable controller 50 does not include any on-board processors and simply includes a circuit board 57 and data and electrical inputs and outputs 54, 56 respectively for connection with the external control 80 and the one of more of the implanted pumps 20, 30. The circuit board 57 includes shunts for delivering power from the electrical input 54 to the outputs 56 and thereby to the one of more of the implanted pumps 20, 30.

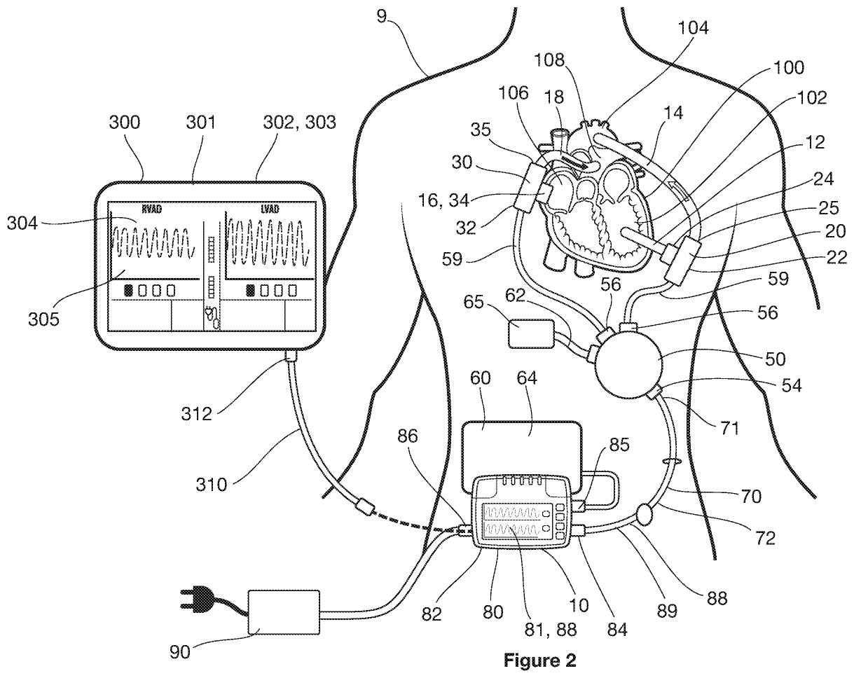

In embodiments, the inputs and outputs 56 of the implantable controller 50 are configured for connection with one or more leads 59 comprising input conductors electrically connected to the pumps 20, 30, including one ore more sensors in the pumps 20, 30 such a pumps speed sensor, or to one or more sensors such as an ECG sensor and an accelerometer surgically implanted on or near the heart 100. The inputs and outputs 56 are also configured for connection with one or more output conductors comprising the one or more leads 59 that are electrically connected to the drive motors of the pumps 20, 30. In FIG. 2, there is shown an embodiment including an implanted battery 65 that is electrically connected by a power lead 62 comprising a conductor that is electrically connected to the circuit board 57 of the implantable controller 50.

The control module, which is comprised of the external controller 80 and the implantable controller 50, controls power supplied from the external battery 60 or the implanted battery 65 to the one or more of the pumps 20, 30 to control the speed of each of the pumps 20, 30 and thereby control the output of each one of the pumps 30. In particular, the one or more of the processors 83, 53 of the implanted controller 50 and/or the external controller 80 are configured to estimate the flow through each one of the pumps 20, 30, preferably in real time, from data received at the one or more of the processors 83, 53. The data from which the estimate of flow is determined can include data indicative of the electrical power provided to each one of the pumps 20, 30 (i.e. the power consumption of the pumps 20, 30) and/or data indicative of the speed of each one of the pumps 20, 30.

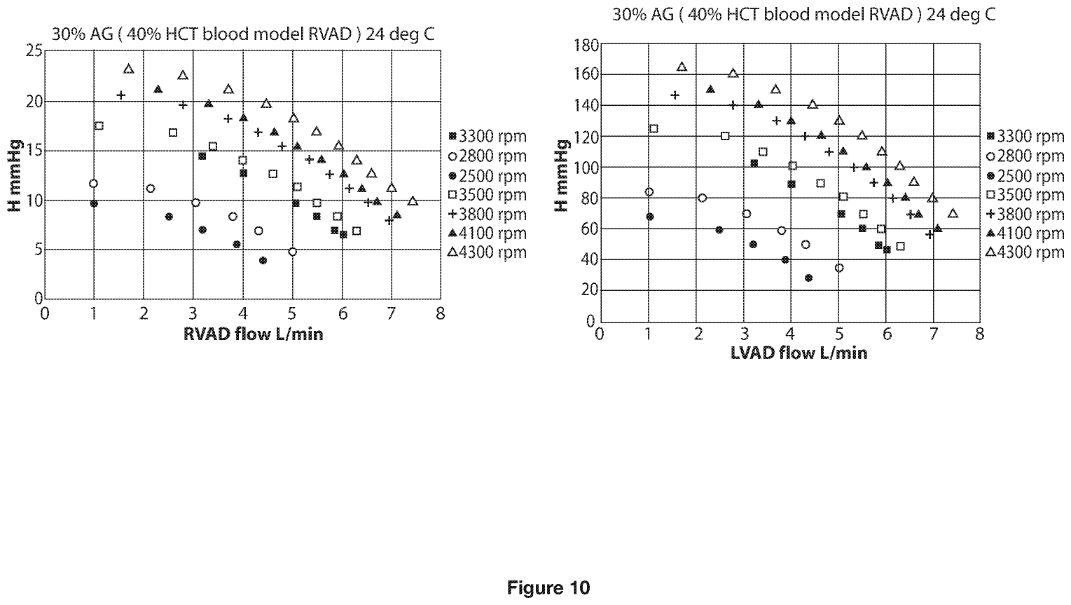

Referring to the graphs in FIG. 10, which illustrates experimental data plotted as pressure versus flow for implanted left and right pumps, it can be seen that the characteristics of an implanted left ventricular assist pump 20 (LVAD) and right ventricular assist pump 30 (RVAD) are different. The right pump 30 generates much lower pressure than the left pump 20 for the same flow rate and same pump speed. This is to match the relative pressures of arterial and venous circulation. Embodiments of the system 1 and the device 10 that provide for the speeds of the pumps 20, 30 to be individually controlled allows some deviation outside of the natural response windows of the pumps 20, 30. If both pumps 20, 30 were set at the same speeds they would produce the same flow rate at different pressures.

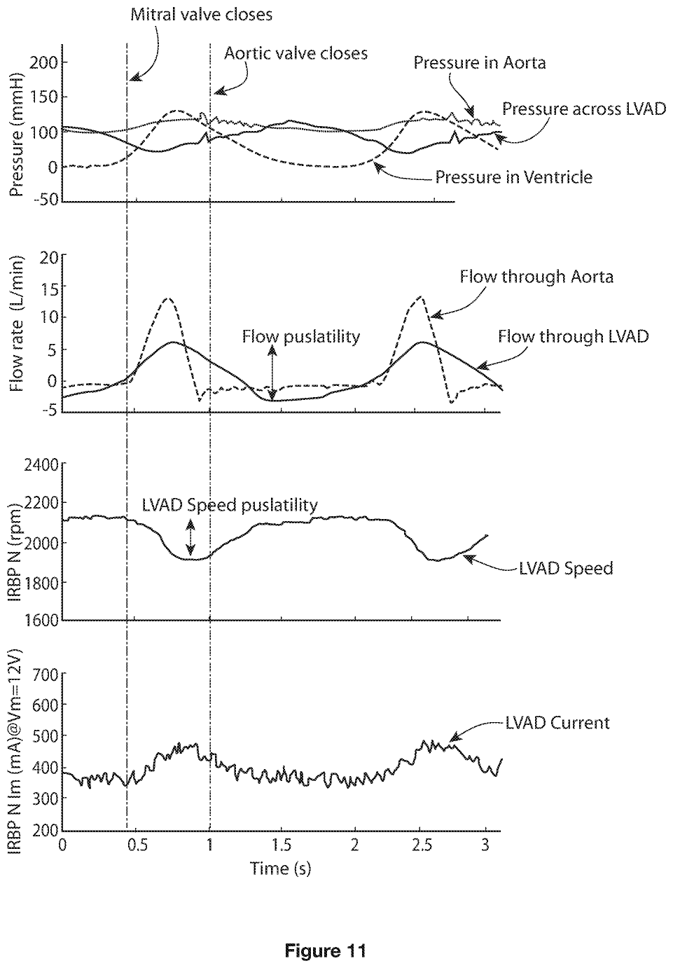

Referring to the graphs in FIG. 11, plotting pressure, flow rate, pump speed and current over time for a left ventricular assist device, the interaction of the left pump 20 and the left ventricle 102 are such that the contractions of the heart 100 cause the speed of the left pump 20 and current (i.e. power) drawn by the left pump to be modulated by these contractions. Dynamically the speed of the left pump 20 falls and flow and current (i.e. power) rises in systole (i.e. ejection) and speed of the left pump 20 increases and flow and current (i.e. power) fall in diastole (i.e. filling) over many cycles as an average. As the left pump 20 speed is increased the heart 100 does less work and contractions are reduced in intensity. The reduction in intensity causes a reduction in flow and hence speed pulsatility of the left pump 20.

Embodiments of the invention monitor the estimated instantaneous flow of the left pump 20 to identify these changes in pump speed and current (i.e. power). The variation of speed pulsatility of the left pump 20 is used as an indicator to set the correct pumping levels for the left pump 20 as governed by the pulmonary and arterial circulatory loads. Typically the left side pressure is about 100 mmHg and right side about 15 mmHg for an adult human.

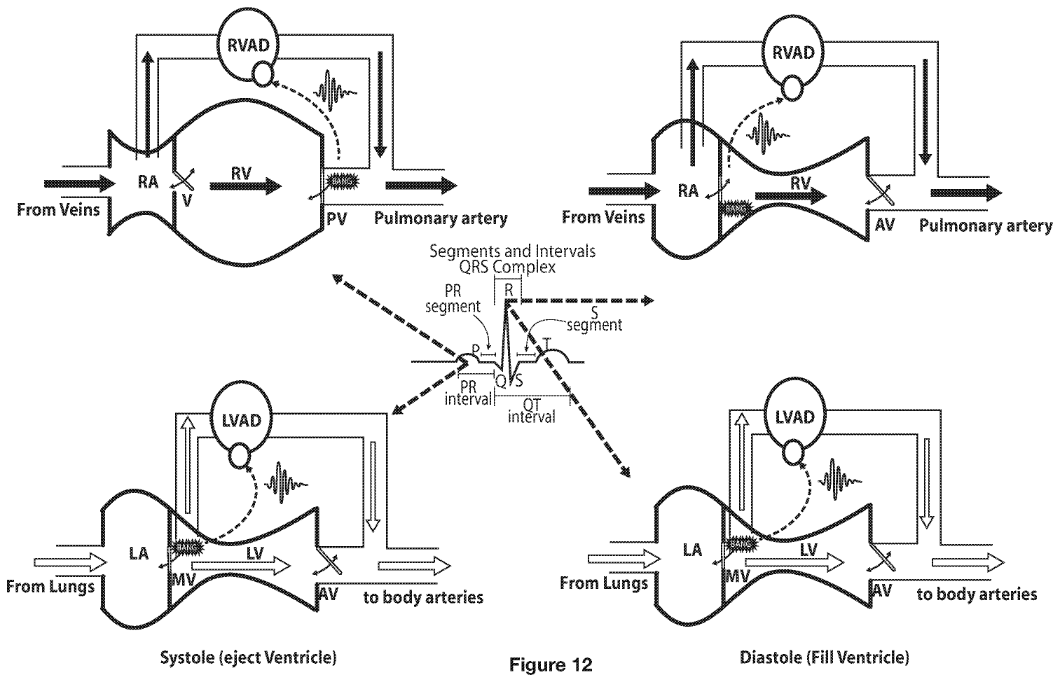

Referring to FIG. 12, there are illustrated schematic representations of the natural electrical impulses of the heart 100 that induce the systole and diastole phases of the heart 100 and the respective contraction and relaxation of the chambers of the heart 100. FIG. 12 also illustrates schematically the operation of the left and right pumps 20, 30 to mechanically assist blood flow for left and right ventricular assistance therapies. FIG. 12 also illustrates schematically how signals from sensors such as an ECG sensor and/or an accelerometer sensor implanted on or near the heart, indicative of the natural electrical activity and/or movement of the heart, is employed in embodiments to assist in facilitating control of the operation of the pumps 20, 30 in a manner that will be described in further detail below.

The control module is configured to execute an algorithm for controlling left pump 20 and right pump 30 flow balance based on the detection and monitoring of speed of the pumps 20, 30 and current (i.e. power) drawn by the pumps 20, 30.

In embodiments, speed of the pumps 20, 30 is determined by reference to back EMF signal detection and processing or hall effect sensors embedded in the pumps 20, 30. The hall effect sensors are disposed in the respective pump housings 22, 32. Current drawn by the pumps 20, 30 can be detected by a current sensor, preferably as a shunt resistor incorporated in the control module (i.e. in the external controller 80 or the implantable controller 50).

The algorithm is configured to estimate the flow rate of the left pump 20 as a function of speed and current of the left pump 20 and to estimate the flow rate of the right pump 30 also as a function of the speed and current of the right pump 30. Using this flow rate estimate, the speed of the left pump 20, in particular, is set such that flow rate has good peak to peak amplitude caused by the native cardiac cycle impressing on the behaviour of the pump 20.

A mathematical model is derived from measurements of flow through the pump with varying electrical power supplied to the pump 20 and/or speeds of the pump 20 and establishing correlations therebetween. The flow is a function of the blood viscosity, the electrical power and/or the speed of the pump 20. Measurements of the speed of the pump 20 and/or the power drawn by the pump 20 can be compared with the mathematical model, preferably using a curve fitting function, to determine an estimate of the flow of the pump 20 when in use to provide ventricular assistance therapy. Likewise the same approach is adopted to determining a mathematical model for flow and for determining estimates of flow for the right ventricular assistance therapy pump 30.

In particular, for a given blood viscosity, the average flow rate of both pumps 20, 30 is expressed as:

LVAD flow estimate (t)=f(Nl(t))+f(Pl(t))

RVAD flow estimate (t)=f(Nr(t))+f(Pr(t))

Where Nl(t)=measured impeller speed of left pump at a time t, Pl(t)=, measured (t) electrical power drawn by left pump at a time t
Where Nr(t)=measured impeller speed of right pump at a time t Pr(t)=measured electrical power drawn by right pump at a time t

Using the flow estimate, the speed of the left ventricular assist device pump 20 is set such that flow has good peak to peak amplitude caused by the native cardiac cycle impressing on the pump 20 behaviour. The flow of the right pump 20 is then set by the following criteria:

Embodiments of the flow balancing algorithm are further configured so that the speed of the right pump 30 is set such that the estimate of the flow rate of the right pump is about 85 to 95 percent, or any increment therebetween or preferably about 90 percent of the flow rate of the left pump 20. In an embodiment, the control module is configured to compare the flow estimates of the left pump 20 and the right pump 30 and to determine if any error exits and to cause an alarm to be issued in a manner described in further detail below.

In embodiments, the control module can include a passive operation mode in which operation of the right pump 30 is passive. In an embodiment, the passive operation includes setting the speed of the right pump 30 to a level at which assistance in driving the flow of blood is nil or minimal while maintaining flow through the left pump 20. The passive operation of the right pump 30 is advantageous in that normal operation of the heart is not hindered and complications such as blood clotting is minimised. Also, where the right pump 30 is of the type that includes a floating impeller, as will be discussed in further detail below, passive operation of the right pump 20 means that bearing stability is maintained. This passive operation mode can be used when the function of the right side of the heart is adequate but the risk of development of right side heart failure is possible.

Referring to the embodiments of FIGS. 1 to 5, the percutaneous lead 70 includes the at least one conductor 72 having a first end 71 electrically connected to the circuit board 57 of the implantable controller 50 and a second end 73 that is located externally of the body of the patient 9 in which the controller 50 is implanted. The lead 70 is implanted percutaneously such that intermediate the first and second ends 71, 73 the lead 70 transits an incision through the skin of the patient 9.

The second end 73 of the percutaneous lead 70 is configured for electrical connection with the external controller 80 and/or directly to the external power supply which includes a rechargeable battery pack 64. A mains power supply connector can be connected to the external controller 80 to recharge the external power supply 60 and also to recharge a reserve power supply contained in the external controller 80. The one or more conductors 72 within the percutaneous lead 70 conduct electrical current from the external power supply 60 either directly or indirectly through the external controller 80 to power the implantable controller 60 and the one or more of the pumps 20, 30 and in certain embodiments to also charge the implanted battery 60. The one or more conductors 72 within the percutaneous lead 70 also conduct electrical signals between the implantable controller 60 and the external controller 80 to facilitate data transfer therebetween.

The input/output 84 of the external controller 80 is configured for connection with an external lead 88 comprising one or more conductors 89 for electrical connection with the at least one conductor 72 of the percutaneous lead 70. Electrical connection of the percutaneous lead 70 and the external lead 88 facilitates conduction of electrical current from the external power supply 90 indirectly through the external controller 80 to power the implanted controller 60 and the pumps 20, 30 and in certain embodiments to also charge the implanted battery 60 and also for conduction of electrical signals between the implanted controller 60 and the external controller 80 to facilitate data transfer therebetween.

Electrical connection of the percutaneous lead 70 and the external lead 88 can be by way of direct physical contact of electrical terminals at the free ends of the leads 70, 88. In this embodiment, the percutaneous lead 70 and the external lead 88 include a releasable coupling to physically hold the electrical terminals in contact with each other. The coupling component at the second or free end 73 of the percutaneous lead 70 includes a water-proof cap to prevent exposure of the terminal to water when the percutaneous lead 70 is not-connected to the external lead 88.

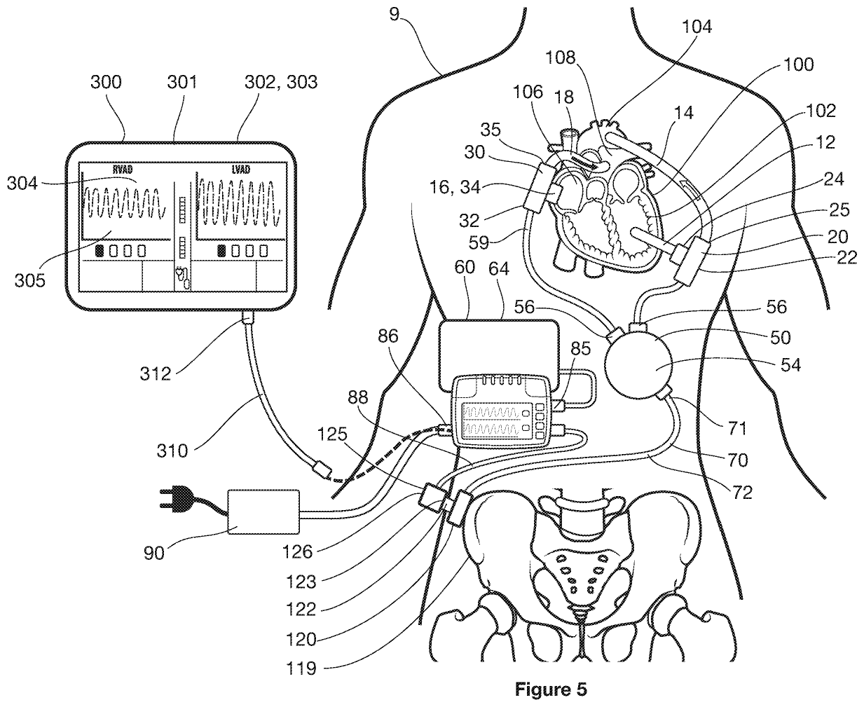

FIG. 5 illustrates an embodiment including a percutaneous iliac crest connection assembly 120 for connection of the at least one conductor 72 of the percutaneous lead 70 and the at least one conductor 89 of the external lead 88 for the supply of power from the external controller 80 and the external power supply 60 and for data transfer with the implantable controller 50.

The implanted lead 70 extends from the implantable controller 50 to an implanted connector 120 that is rigidly connected to the iliac crest of the pelvis 119. The implanted connector 120 has a base portion 121 that is rigidly fixed to the iliac crest by one or more bone screws. The implanted connector 120 includes an elongated percutaneous portion 122 that is upstanding from the base portion 121. An electrical contact 123 is incorporated in a distal end of the elongated percutaneous portion 122 and thereby provides a stable, external electrical contact 123 that protrudes out of the patient's hip. The conductor 72 within the lead 70 is electrically connect, either directly or indirectly to the external electrical contact 123.

The external lead 88 which has one end connected to the external controller 80 has an opposite end comprising a fitting 125 containing an electrical contact 126 for connection with the distal end of the elongated percutaneous portion 122. The fitting 125 includes a locking mechanism, including a two-step unlocking process, for connecting with the distal end of the elongated percutaneous portion 122, and for reducing the risk of inadvertent disconnection. Because the elongated percutaneous portion 122 that is upstanding from the base portion 121 is very securely and rigidly fixed to the iliac crest, any movement relative to the skin surrounding the penetration is minimised which is beneficial for reducing risk of infection or other complications.

In another embodiment, not illustrated in the figures, connection of the at least one conductor 72 of the percutaneous lead 70 and the at least one conductor 89 of the external lead 88 is provided wirelessly. A percutaneous lead side induction coil is electrically connected to the second end 73 of the conductor 72 of the percutaneous lead 70 for wireless coupling of the external control module 80 and power supply module 60 to the implantable controller 50 and the pumps 20, 30 for the supply of power thereto and for wireless data transfer. In an embodiment, data transfer is achieved by modulating the power supplied to the implantable controller 50. An external lead side induction coil is connected to a free end of the conductor 89 of the external lead 88.

The induction coils are each contained within respective housings. The housings are configured to include locating means in the form of complementary shaped housing sections and embedded magnets. The locating means are adapted to be brought together and to locate the induction coils within the respective housings relative to each other to establish and inductive coupling therebetween. The inductive coupling employs electromagnetic induction to induce current to supply power to the implantable controller 50 and the implanted pumps 20, 30 and, in certain embodiments, to charge the implanted battery 60 and to facilitate data transmission between the implantable controller 50 and the external controller 80.

The housing encasing the percutaneous lead side induction coil is durable and water-tight at the second or free end 73 of the percutaneous lead 70 to prevent exposure of the coil to water.

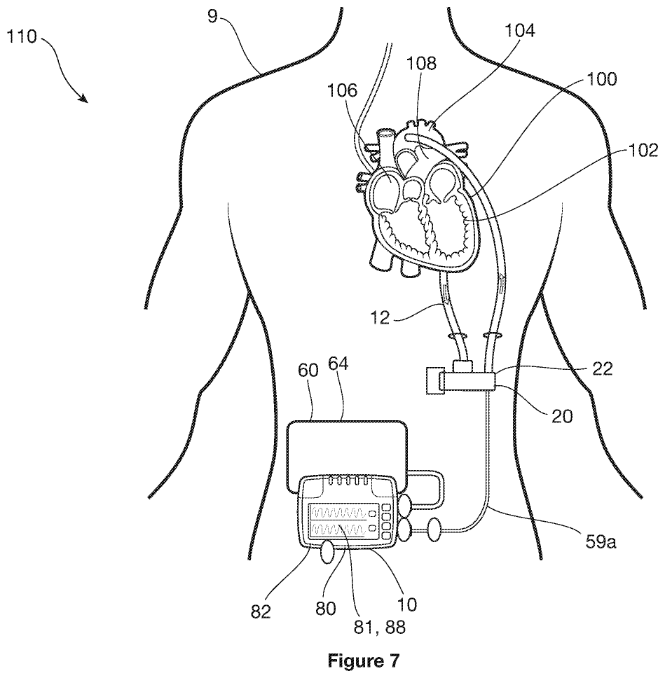

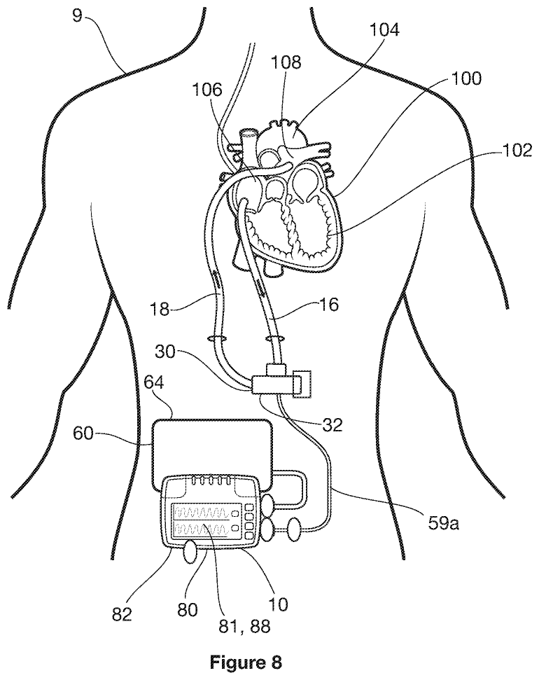

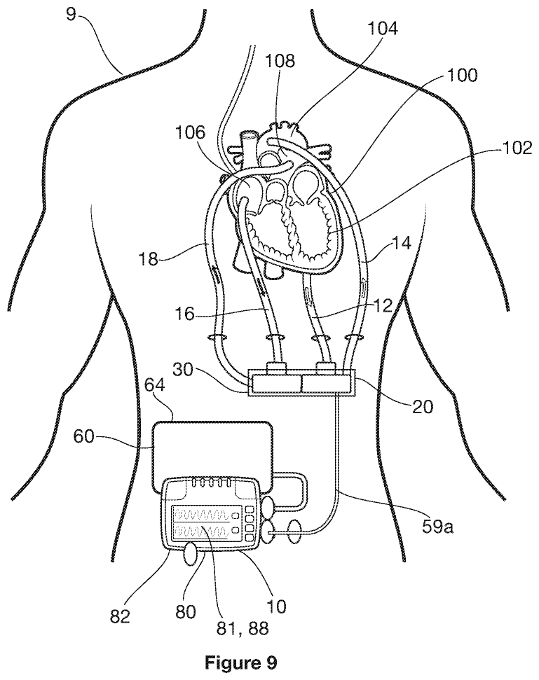

FIGS. 7 to 9 illustrate diagrammatic illustrations of a variety of embodiments of a left and/or right ventricular assist devices 110 in which the pumps 30 are located externally. These embodiments comprise an arrangement in which the two pumps 20, 30 are contained in separate housings 22, 32. FIG. 9 illustrates a biventricular assistance therapy configuration in which the two housings 22, 32 of the pumps 20, 30 are coupled together with a connector 40 or are integrally formed in a single housing. FIG. 7 illustrates a left ventricular assistance therapy configuration including a single one of the pumps 20. FIG. 8 illustrates a right ventricular assistance therapy configuration also including a single one of the pumps 30.

In contrast to other embodiments in which the pumps 20, 30 and the module 50 and battery 60 are implanted, in the embodiments of FIGS. 7 to 9 these components are located externally and the inlet tubes 12, 16 and the outlet tubes 14, 18 are implanted percutaneously. Such arrangements are adapted for use where low cost or temporary assistance is required and in which the components can be formed out of relatively low cost materials.

In the embodiments of FIGS. 7 to 9, the control module is comprised of the external controller 80. The external controller 80 controls power supplied from the external battery 60 to the one or more of the pumps 20, 30 via leads 59a connected therebetween to control the speed of each of the pumps 20, 30 and thereby control the output of each one of the pumps 20, 30. In particular, one or more of the processors 83 are configured to estimate the flow through each one of the pumps 20, 30, preferably in real time, from data received at the one or more of the processor 83. The data from which the estimate of flow is determined can include data indicative of the electrical power provided to each one of the pumps 20, 30 (i.e. the power consumption of the pumps 20, 30) and/or data indicative of the speed of each one of the pumps 20, 30, such as may be determined with back EMF signal detection and processing or from signals received from hall effect sensors mounted in the respective pump housing 22, 32.

The external controller 80 is flexibly adapted to control the configurations of the device 10 of FIGS. 1 to 5, which include implanted pumps 20, 30 in left ventricular assistance or right ventricular assistance or bi-ventricular assistance therapy configurations, and including an implanted controller 50 including some or no on-board processing capability. The same external controller is flexibly adapted to control the configurations of the device 110 of FIGS. 7 to 9, which include external pumps 20, 30 in left ventricular assistance or right ventricular assistance or bi-ventricular assistance therapy configurations.

In embodiments, the external controller 80 is flexibly adapted to control a catheter-based miniaturized ventricular assist device. Catheter-based ventricular assist devices are placed in the left ventricle across the aortic valve via femoral artery access. The device pumps blood from the left ventricle into the ascending aorta and helps to maintain a systemic circulation at an upper rate between 2.5 and 5.0 L/min. This results in almost immediate and sustained unloading of the left ventricle, while increasing overall systemic cardiac output. Catheter-based ventricular assist devices are typically used as a temporary therapy, such as in the treatment of acute myocardial infarction.

The external controller 80 is programmable either directly via the user interface 88 or via a remote computing platform operated by a cardiologist, to connect via the input/output 84 to a lead that includes a conductor connected to the catheter-based miniaturized ventricular assist device and thereby control the operation of the device. The catheter-based miniaturized ventricular assist device includes an on-board electrically powered pump that is adapted to be powered and controlled in a similar manner to the pumps 20, 30 described above. Accordingly, the same external controller 80 can be flexibly employed with catheter-based miniaturized ventricular assist devices.

FIGS. 1 to 5 illustrate embodiments of the heart assist system 1 including a remote monitoring platform 300. The remote monitoring platform 300 comprises a device 301 which has a processor 302, a storage medium 303, a display 304 and a user interface 305 such as a keyboard or touchscreen. The device 301 also includes a receiver 312 to receive signals from the external controller 80 of the heart assist device 10 providing the patient 9 with left and/or right ventricular assistance therapy. The external controller 80 may be configured to transmit and receive data to the remote monitoring device 301 by a wired connection therebetween by a data cable 310 or wirelessly. In embodiments wireless communication is by Wi-Fi, Bluetooth or via a mobile telecommunications network.

The remote monitoring platform 300 embodiment enables a clinician to remotely monitor the operation of the heart assist device 10, to analyse biometric data from the patient such as ECG data and any other data collected by the implantable controller 50 and/or by the external controller 80 such as stored data in relation to left and/or right pump 20, 30 speed and current draw parameters. The remote monitoring system 300 may also be configured to receive alerts from the external controller 80 or the implantable controller 50 indicating abnormal operation of the device 10 or biometric data indicating that acute clinical intervention is required.

A clinician can make changes to the settings in the external controller 80 or the implantable controller 50 in relation to various control parameters such as the output of the left pump 20 and/or the right pump 30. Other settings that can be changed include in relation to the balance of the relative output of the pump 20 and the right pump 30.

In embodiments, the remote monitoring platform 300 further includes a data connection between the device 301 and a device controlled by a remotely located clinician or hospital. Communication between the device 301 and the remotely located clinician or hospital is via wired or wireless communications network including the Internet and/or via a mobile telecommunications network.

The remote monitoring platform 300 embodiments enable rapid diagnosis of clinically significant information and similarly rapid adjustments to the heart assistance therapy being provided to the patient 9 by the device 10.

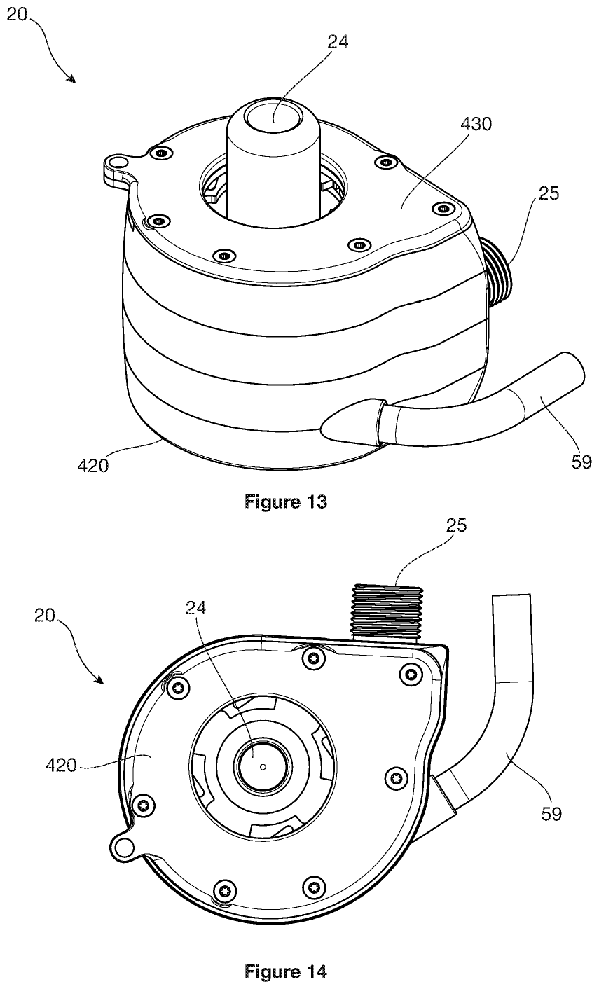

FIGS. 13 to 20 illustrate embodiments of the left pump 20 and the right pump 30. The left pump 20 and the right pump 30 are identical and are formed separately and, in some embodiments are optionally connected together, to provide for a variety of configurations including left ventricular assistance only, right ventricular assistance only or left and right ventricular assistance where the left and right pumps 20, 30 are co-located or are located separately to each other.

For convenience, the following description of components of only one of the pumps of the configurations of the device 10 of FIGS. 1 to 9 is provided, namely of the left pump 20. However, it is to be appreciated that the right pump 30 will be similarly configured and where any differences exist these will be described.

The pump 20 is a centrifugal blood pump and as previously described, the housing 22 of the pump 20 includes an inlet opening 24 and an outlet opening 25. The housing 22 is formed out of a pair of moulded sections 420, 430 that when brought together define an internal chamber 440. The chamber 440 houses an impeller 450 and is shaped to cooperate with the impeller 450 in a manner described in further detail below. In embodiments, the pump 20 is configured as axial flow or a radial flow or a hybrid flow centrifugal pump.

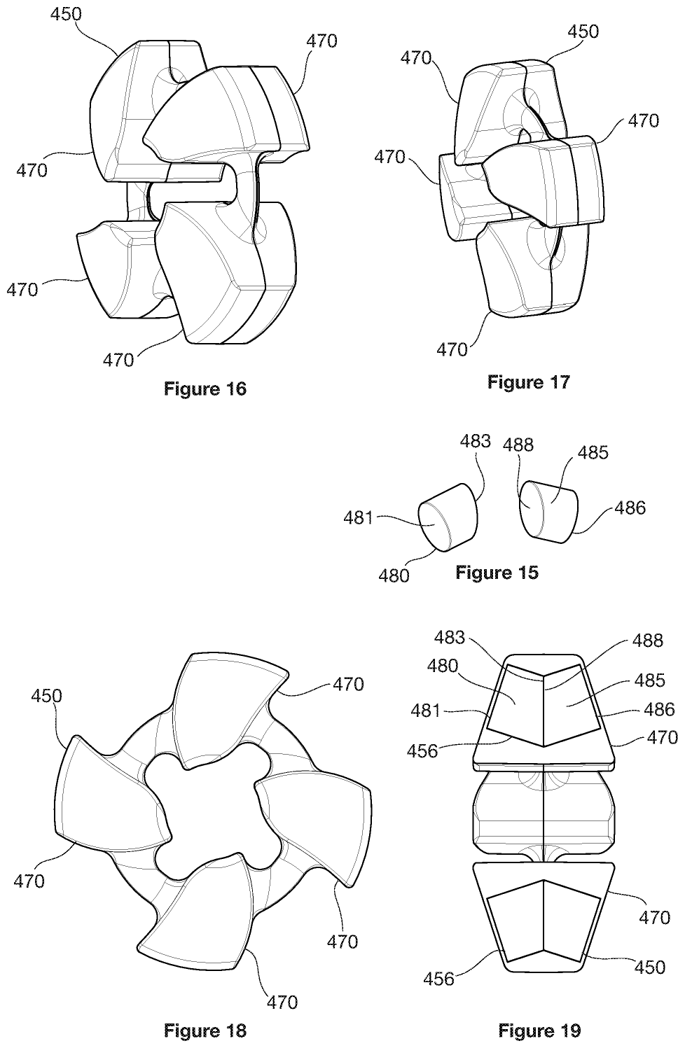

FIGS. 15 to 20 illustrate an embodiment of the impeller 450 and the method of manufacture thereof. In the assembled pump 20 the impeller 450 is the only moving part. The impeller 450 is adapted to a rotating impeller which ‘floats’ in the blood, reducing wear and potential for blood clots. The impeller 450 is electromagnetically driven impeller containing magnets 480, 485 sealed inside blades 470 of the impeller 450.

The impeller 450 is sealed within the chamber 440 by an inwardly facing wall 445 of the housing 22. The impeller 450 is suspended or floating within the chamber 440 and is not supported or maintained in axial alignment by a shaft or the like. The housing 450 contains one or more coils 460, 465 electrically connected to the conductor 59 which, as described above, turn is electrically connected to the implantable controller 50 and indirectly to the external controller 80 and the to the battery 60. A supply of current to the coils 460, 465 from the battery 60 induces an electrical field that is experienced by the magnets 480, 485 inside the impeller blades 470. Electromagnetic torque is provided by the interaction between the magnets 480, 485 embedded in the blades 470 and the electromagnetic field generated by the coils 460, 465. The current supplied to the coils 460, 465 is an alternating current so as to induce the coils to generate a rotating current pattern coils fixed relative to the housing 22. The electromagnetic torque induces rotation of the impeller 450 relative to the stationary housing 22.

The impeller 450 is suspended within the housing 22 by hydrodynamic forces from interaction between the rotating impeller 450, the internal wall 445 of the housing 22 and the blood that the impeller 450 moves from the inlet 24 to the outlet of the housing 22. The shape of the blades 470 of the impeller 450 are such that blood entering the chamber 440 from the inlet 24, which is arranged axially, is driven out through the outlet 24, which is offset or tangential to the axis of rotation of the impeller 450.

FIGS. 15 and 19 illustrate magnets 480, 485 to be sealed in the impeller blades 470. The material from which the impeller casing 455 containing the magnets 480 is formed is a titanium alloy. The impeller casing 455 is formed out of two cast pieces of titanium alloy with a plurality of cavities 456 for receiving the magnets 480, 485 therewithin. Each cavity 456 is sealed by bringing together and fusing the cast pieces.

Referring to FIG. 19, each one of the cavities 456 of the casing 455 is adapted to receive a pair of the magnets 480, 485 of FIG. 15 stacked one upon another. To achieve an optimal magnet configuration within the impeller blades 470, each magnet 480, 485 is obtained from the same base material but engineered into two discrete magnet parts 480, 485. One of the magnet parts 480 is formed substantially cylindrically and axially aligned with its dipole. One of the end faces 481 will be oriented perpendicularly to the dipole whereas the opposite end face 483 will be oriented at an inclined or canted angle relative to the dipole (e.g. between 5 and degrees and preferably between 10 and 20 degrees).

The other magnet 485 has an identical external form to the first magnet 480 but with an opposite polarity. In other words, the other magnet 485 is formed substantially cylindrically and axially aligned with its dipole. One of the end faces 486 is oriented perpendicularly to the dipole, however, the polarity of this perpendicular end will be opposite to that of the first magnet 480. The opposite end face 488 will be oriented at an inclined or canted angle relative to the dipole (e.g. between 5 and 30 degrees and preferably between 10 and 20 degrees).

The first and the second magnets 480, 485 are assembled in the impeller cavity 456 so that the inclined or canted angle surfaces 483, 488 are in face to face contact and the opposite perpendicular end faces 481, 486 face away from each other and are opposite in polarity and inclined relative to the axis of rotation of the impeller 450. One set of electric coils 460 are embedded in or attached to one of the pair of moulded sections 420 that forms a base 422 of the chamber 440 and the other set of electric coils 465 are embedded in or attached to the other one of the pair of moulded sections 430 that forms a cover 432 of the chamber 440, and that together with the base 422 forms the enclosed chamber 440. The resulting impeller 450 includes a configuration of embedded magnets 480, 485 that are optimally configured to interact with the angularly offset coils 460, 465 that are arranged in the base 422 and the cover 432 of the chamber 440. The dipoles of the magnets 480, 485 are optimally positioned and oriented with respect to the electric coils 460, 465.

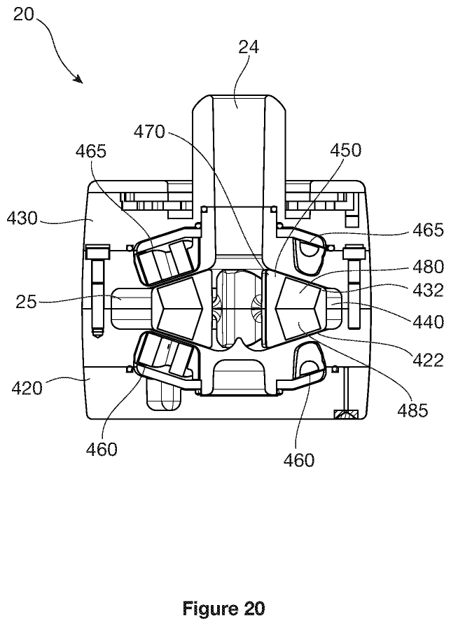

The impeller 450 includes four blades 470 that each contain a set of the first and the second magnets 480, 485 in the configuration described above and illustrated in FIGS. 19 and 20, however, the magnets 480, 485 in adjacent blade 470 are oriented with opposite polarities. In embodiments, the resulting impeller 450 and the blades 470 thereof are symmetrical about a plane transverse to the axis of rotation.

It is critical that the magnets 480 comprised of the magnet parts 480, 485 are hermetically sealed within the cavities 456. Embodiments of the invention, call for a seal to be achieved by laser welding or by mechanically deforming specifically provided excess casing material adjacent to a joint to be sealed. The small excess of titanium, or other material used to form the casing 455 of the impeller 450, is deliberately engineered adjacent to the joint to be sealed in the form of a reverse taper.

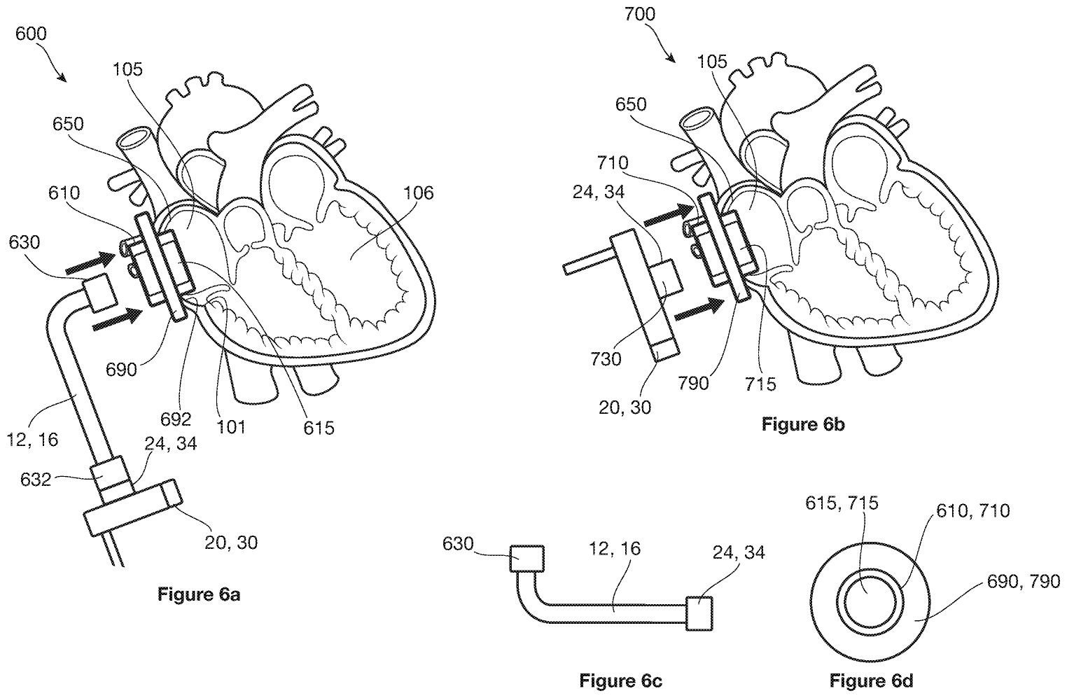

Referring to FIGS. 6a, 6c and 6d, there is shown an embodiment of a connector assembly 600 for connecting a cannula, hereinafter referred to as a tube 12, 16 extending from the flow inlet 24, 34 of one of the blood pumps 20, 30 to a chamber of the heart 100. Referring to FIGS. 6a and 6d, there is shown an embodiment of the connector assembly 600 including a female fitting 610 for implantation within an opening through a wall 650 of a heart chamber, such as the right atrium 105, as illustrated in FIG. 6a, or the left ventricle 106. The female fitting 610 includes an axial passage 615 for receiving a male fitting 630 connected to an end of the inlet tube 12, 16, as illustrated in FIGS. 6a and 6c, that includes a pump fitting 632 at an opposite end that is coupled to a left pump 20 or right pump 30. The male fitting 630 is adapted to be locked within the axial passage 615 of the female fitting 610.

The male fitting 630 and the female fitting 610 include a modified bayonet-type connection to securely fasten the male and female fittings 630, 610 together. In another embodiment, the male and female fittings 630, 610 are sutured together.

Referring to FIG. 6a, the female fitting 610 is fitted within a felt collar 690 sewn felt onto the heart 100 immediately adjacent to or within a hole 692 bored through the heart wall 101. The female fitting 610 is fitted tightly within the felt collar 690 to thereby provide fluid communication between axial passage 615 of the female fitting 610 and the right atrium 105 or the left ventricle 102. Alternatively, the female fitting 610 and the felt collar 690 may be fixed or fused together.

The embodiment of FIG. 6a, which employs elongated tubes 12, 16 extending from the flow inlet 24, 34 of one of the blood pumps 20, 30 to the connector assembly 600 in fluid communication with a chamber of the heart 100 permits indirect connection of the blood pumps 20, 30 to the left ventricle 106 and right atrium 105 respectively. Thus, the pumps 20, 30 can be positioned in a suitable location distally from the heart.

FIG. 6b illustrates another embodiment of the connector assembly 700 for connecting directly to the flow inlet 24, 34 of one of the blood pumps 20, 30 and to a chamber of the heart 100. The connector assembly 700 includes a female fitting 710 for implantation within an opening through a wall 650 of a heart chamber, namely of the right atrium 105 or the left ventricle 106. The female fitting 710 includes an axial passage 715 for receiving a male fitting 730 connected directly to or comprising the inlet 24 of the left pump 20 or right pump 30. Preferably, an o-ring is located between the contacting ends of the male and female fittings 730, 710. A clamp 701 is provided to lock the ends of the male and female fittings 730, 710 together.

The invention may be susceptible to other modifications or mechanical equivalents without departing from the spirit or ambit of the invention disclosed herein.

1. A heart assist device comprising:
one or more blood pumps for connection to a heart;
a control module for controlling operation of the one or more pumps;
a power module for powering the operation of the control module and the one or more pumps; and
a lead for supplying power between the control module and the one or more pumps.

2. The device of claim 1, wherein at least part of the control module is located externally and the one or more pumps are implantable in the body and the lead extends percutaneously between the external part of the control module and the one or more implanted pumps.

3. (canceled)

4. (canceled)

5. The device of claim 1, wherein the control module is operable to estimate the flow through each of the one or more pumps from data received at the control module, which data is indicative of electrical power provided to each of the one or more pumps and/or a speed of each of the one or more pumps.

6. The device of claim 1, wherein the control module is configured to control the speed of each of the one or more pumps according to the estimate of flow through each of the one or more pumps.

7. The device of claim 1, wherein the control module controls the speed of each of the one or more pumps to increase the output of each of the one or more pumps during the systole phase of the cardiac cycle and to reduce the output of each of the one or more pumps during the diastole phase of the cardiac cycle.

8. The device of claim 1, wherein the control module is configured to control the speed of each of the one or more pumps to balance the flow through the left and right ventricles.

9. The device of claim 1, wherein the control module is configured to set a target speed for a one of the pumps associated with the right ventricle to generate a flow equivalent to a proportion of the flow of a one of the pumps associated with the left ventricle.

10. The device of claim 9, wherein the proportion is between 85 and 95 percent or any increment therebetween or is preferably 90 percent.

11. The device of claim 1, wherein the power module includes at least one battery that is connected to the control module.

12. The device of claim 11, wherein the power module includes at least one external battery and an implanted battery.

13. The device of claim 1, wherein the lead includes an implanted portion connected to the one or more pumps and an external portion connected to an external part of the control module and an electrical connection between the implanted and external portions of the lead.

14. The device of claim 13, wherein the electrical connection includes an implanted connector that is rigidly connectable to the iliac crest of the pelvis and that is adapted for electrical connection with an external connector that is connected to the external portion of the lead.

15. The device of claim 14, wherein the implanted connector has a percutaneous portion that passes through the skin and an external electrical contact for connection with an electrical contact of the external connector.

16. (canceled)

17. (canceled)

18. The device of claim 1, including a connector assembly for connecting a flow inlet of one of the blood pumps to a chamber of a heart
wherein the connector assembly includes a first fitting for implantation within an opening through a wall of a heart chamber, the first fitting including an axial passage for receiving a second fitting connected to the flow inlet of the one or more blood pumps,
wherein the second fitting is connected indirectly to the flow inlet of the one of the blood pumps with an elongated conduit for locating the pump in a suitable location distally from the heart.

19. (canceled)

20. (canceled)

21. The device of claim 18, wherein the second fitting is connected directly to a housing of the one of the blood pumps at the flow inlet for locating the pump immediately adjacent to the heart,
wherein the first fitting is mounted within a cuff adapted to be sutured into place circumferentially within the opening in the heart wall,
and where the cuff is formed of a surgical felt material.

22. (canceled)

23. (canceled)

24. (canceled)

25. (canceled)

26. (canceled)

27. (canceled)

28. (canceled)

29. A control system for a ventricular assistance device that includes one or more blood pumps for providing left and/or right ventricular assistance therapy, the system including:
a control module including a processor for receiving data indicative of electrical power provided to the pump and/or a speed of the pump;
estimating the flow through each of the one or more of the pumps from the data received at the processor by comparing the received data with a mathematical model indicative of the relationship between the electrical power supplied to the pump and/or the speed of the pump and the flow through the pump.

30. The control system of claim 29, wherein the mathematical model is derived from measurements of flow through each of the one or more pumps with varying electrical power supplied to the pump and/or speeds of the pump.

31. (canceled)

32. The control system of claim 29, wherein the processor is configured to control the output of the pumps to balance the flow through the left and right ventricles,
wherein the processor is configured to set a target speed for the one of the pumps associated with the right ventricle to generate a flow equivalent to a proportion of the flow of the other one of the pumps associated with the left ventricle, wherein the proportion is preferably between 85 and 95 percent or any increment therebetween or 90 percent.

33. (canceled)

34. A method for controlling a ventricular assistance device that includes one or more blood pumps for providing left and/or right ventricular assistance therapy, the method including:
receiving at a processor of a control module data indicative of electrical power provided to the one or more of the pumps and/or a speed of the one or more of the pumps;
estimating the flow through each of the one or more pumps by comparing the received data with a mathematical model indicative of the relationship between the electrical power supplied to the pump and/or the speed of the pump and the flow through the pump; and
controlling the output of the pumps by adjusting the electrical power provided to the pumps.

35. (canceled)

36. (canceled)

37. The device of claim 2, wherein at least part of the control module is located internally and the one or more pumps are implantable in the body and the lead extends percutaneously between the external part of the control module and the one or more implanted pumps via the internal part of the control module.

38. The device of claim 1, wherein the control module for controlling the operation of the one or more pumps is located internally and the one or more pumps are implantable in the body and wherein power is provided percutaneously between an external power supply and the control module.
